(19) United States
(12) Patent Application Publication
(10) Pub. No.: US 2015/0307538 A1
(43) Pub. Date: Oct. 29, 2015

(54) USE OF THERMALLY-TREATED SUPPORTED COBALT CATALYSTS COMPRISING A POLYCYCLIC AROMATIC STRUCTURE CONSISTING OF NITROGEN LIGANDS FOR HYROGENATING AROMATIC NITRO COMPOUNDS

(71) Applicant: EVONIK INDUSTRIES AG, Essen (DE)

(72) Inventors: Felix Westerhaus, Mannheim (DE); Rajenahally Venkataswamygowda Jagadeesh, Bangalore (IN); Gerrit Wienhofer, Essen (DE); Annette-Enrica Surkus, Rostock (DE); Kathrin Junge, Rostock (DE); Henrik Junge, Rostock (DE); Matthias Beller, Nienhagen (DE)

(21) Appl. No.: 14/405,963

(22) PCT Filed: Jun. 7, 2013
(86) PCT No.: PCT/EP2013/061757

§ 371 Date: May 22, 2015

(30) Foreign Application Priority Data
Jun. 8, 2012 (DE) 10 2012 209 634.5

(51) Int. Cl. C07F 15/06; C07C 319/20; C07D 311/14; B01J 37/08; C07D 277/64; C07D 215/40; B01J 31/18; C07C 209/36; C07D 213/73

(52) U.S. Cl. CPC C07F 15/065; C07C 209/36; C07C 209/365; C07C 319/20; C07D 311/14; C07D 213/73; C07D 277/64; C07D 215/40; B01J 31/183; B01J 37/086; C07C 2103/12; B01J 2231/641; B01J 2531/845

(57) ABSTRACT

The invention relates to the use of thermally-treated supported cobalt catalysts for hydrogenating aromatic nitro compounds, the cobalt catalysts having been prepared by in situ immobilization of a cobalt-amine complex on an inorganic porous support and subsequent pyrolysis, and, in the cobalt-amine complex used, cobalt being present bonded to an aromatic or heterocyclic nitrogen ligand L, the nitrogen ligand being selected so as to form a polyaromatic structure with the cobalt atom.

The invention relates to the use of thermally-treated supported cobalt catalysts for the selective hydrogenation of aromatic nitro compounds to the corresponding aromatic amines. The invention also relates to novel thermally-treated supported cobalt catalysts and preparation thereof.

Aromatic amines, such as aniline and derivatives thereof, are valuable intermediates for the production of polymers, fine chemicals, agrochemicals and pharmaceuticals. The preparation of said amines is generally carried out by reduction of the corresponding aromatic nitro compounds, which is effected using reducing agents such as Fe, Zn, Sn, Al and sulfur compounds, electrochemical methods or catalytic hydrogenation. Catalysis is a key technology for favorable processes in the chemical, pharmaceutical and materials industry. Owing to their stability, easy separation and the possibility of recycling, heterogeneous catalysts form an important basis for controlling chemical reactivity. The most environmentally friendly and cost-effective reducing agent is hydrogen, since only water is produced as waste product. Of major interest, therefore, is novel cost-effective, active and selective catalysts for catalytic hydrogenation.

Many of the aromatic amines which are of interest both for organic synthesis and for industry are substituted with a multiplicity of different functional groups. A generally applicable catalyst system is therefore required which allows highly chemoselective reduction of the nitro group of the corresponding starting aromatic compounds. With respect to the selectivity, the reduction of NO₂ in the presence of other reducible groups, such as halogens, ketones, aldehydes, alkenes or alkynes, remains especially difficult. The commercially available Raney nickel, palladium or platinum catalysts are problematic. Using platinum catalysts, although Siegrist et al. and Blaser et al. achieved the selective reduction of substituted aromatic nitro compounds in the presence of specific additives [a) Siegrist, U., Baumeister, P., Blaser, H.-U. Catalysis of Organic Reactions, F. Herkes, Ed., vol. 75 of Chemical Industries Dekker, New York (1998); b) Raja, R., Golovko, V. B., Thomas, J. M., Berenguer-Murcia, A., Zhou, W., Xiee, S., Johnson, B. F. G. Chem. Commun. 2026-2028 (2005); Blaser, H.-U., Siegrist, U., Steiner, H. in Aromatic Nitro Compounds: Fine Chemicals through Heterogeneous Catalysis, Sheldon, R. A., van Bekkum, H. (Eds) Wiley-VCH, Weinheim Germany (2001), Blaser, H.-U., Steiner, H., Studer, M. ChemCatChem 1, 210-221 (2009)], disadvantages, however, are the accumulation of hydroxylamines and a decreasing catalyst performance. Furthermore, a heterogeneous gold-based catalyst is known from the group of Corma, which allows the selective hydrogenation of nitro compounds in the presence of a number of functional groups (olefines, aldehydes, amides) [Corma, A.; Serna, P. Science 313, 332-334 (2006); Corma, A., Gonález-Arellano, C., Iglesias, M., Sánchez, F. Appl. Catal. A: Gen. 356, 99-102. (2009) Corma, A.; Serna, P.; Concepción, P.; Calvino, J. J. Am. Chem. Soc. 130, 8748-8753 (2008)].

Non-volatile organometallic complexes of iron and cobalt have been described in WO 2010/051619 A1 as precursors for heterogeneous catalysts for the reduction of oxygen in fuel cells.

So far, however, no cost-effective catalyst system for the hydrogenation of aromatic nitro compounds is known which does not require precious metals and is generally suitable for the hydrogenation of substituted aromatic nitro compounds. All known methods have the disadvantage that the hydrogenation proceeds insufficiently selectively and the catalysts exhibit low activities and/or have precious metals.

It has been found that molecularly defined cobalt-amine complexes on a heterogeneous support and following a thermal treatment (pyrolysis) are highly selective catalytic materials for the hydrogenation of aromatic nitro compounds to the corresponding aromatic amine derivatives. Surprisingly, the catalyst systems used in accordance with the invention are tolerant to all functional groups on aromatic nitro compounds.

In the context of the invention, “aromatic nitro compounds” are understood to mean substituted and unsubstituted nitrobenzenes and also substituted and unsubstituted heterocyclic aromatic nitro compounds. The aromatic nitro compounds comprise one or more functional groups having unsaturated carbon-carbon, carbon-nitrogen and/or carbon-oxygen bonds on substituents of aromatic nuclei and also halogens (F, Cl, Br, I) and halogen-carbon compounds.

The catalyst systems used according to the invention are prepared in situ from cobalt precursor catalysts, immobilized on an inorganic support and then subjected to thermal treatment (pyrolysis).

These cobalt precursor catalysts are cobalt-amine complexes, cobalt being present bonded to aromatic or heterocylclic nitrogen ligands. They are obtained, for example, by reacting cobalt salts with aromatic or heterocyclic nitrogen ligands (L) and thus form the non-volatile organocobalt complexes (Co-L). The nitrogen atoms which are linked to the cobalt remain associated with said cobalt and thus form the precursor complex. The interactions between Co and ligand L provide the opportunity to modify the form, the electronic and chemical properties of the cobalt-amine complexes. Aromatic nitrogen ligands (L) are known to those skilled in the art. In the context of the invention, all aromatic nitrogen compounds which form a polyaromatic structure with the cobalt atom are suitable as nitrogen ligands.

These are preferably ligands from the group of the phenanthrolines with the general formula:

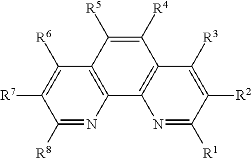

in which R¹ to R⁸ are identically or differently hydrogen, C1-C6-alkyl, C1-C6-Oalkyl, amino, carboxy, halogen, substituted and unsubstituted aryl, substituted and unsubstituted hetereoaryl, preferably selected from the group comprising Me, Et, OMe, NH₂, COOH, phenyl, F, CI and Br, etc. or

pyridines such as terpyridine, 2,6-bis(benzimidazolyl)pyridine, 1,1′-bipyridine and pyridine.

Particular preference is given to nitrogen ligands selected from the group comprising: L1: 1,10-phenanthroline (C₁₂H₁₀N₂), L2: terpyridine (C₁₅H₁₁N₃), L3: 2,6-bis(benzimidazolyl)pyridine (C₁₉H₁₃N₅), L4: 1,1′-bipyridine (C₁₀H₈N₂) and L5: pyridine (C₅H₅N),

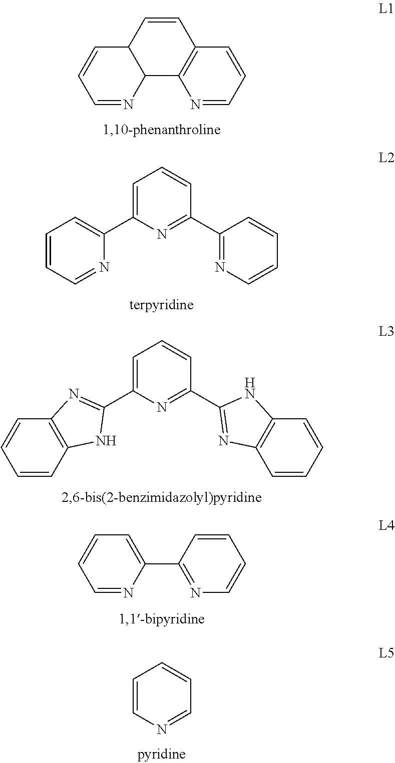 [SMILES: C1=CC2C=Cc3cccnc3C2N=C1 ; c1ccc(-c2cccc(-c3ccccn3)n2)nc1 ; c1cc(-c2nc3ccccc3[nH]2)nc(-c2nc3ccccc3[nH]2)c1 ; c1ccc(-c2ccccn2)nc1 ; c1ccncc1]

and also the 1,10-phenanthroline derivatives L1b: 2,9-dimethyl-4,7-diphenyl-1,10-phenanthroline, L1c: 4,7-dimethoxy-1,10-phenanthroline and L1d: 2,9-dimethyl-1,10-phenanthroline.

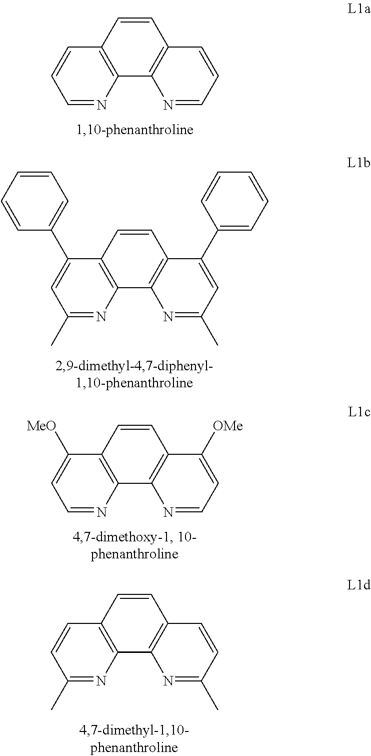 [SMILES: c1cnc2c(c1)ccc1cccnc12 ; Cc1cc(-c2ccccc2)c2ccc3c(-c4ccccc4)cc(C)nc3c2n1 ; COc1ccnc2c1ccc1c(OC)ccnc12 ; Cc1ccc2ccc3ccc(C)nc3c2n1]

The cobalt precursor catalysts are prepared by methods known per se, e.g. by reacting a cobalt salt such as cobalt(II) acetate tetrahydrate with the respective nitrogen ligand in a solvent.

Oxides such as TiO₂ or Al₂O₃ may also be used as inorganic supports in addition to carbon. However, carbon is particularly preferred in various modifications (graphite, graphene, nanotubes) and preparations (pellets, powder, amorphous carbon black, finely dispersed carbon). A support preferably used (e.g. Vulcan XC72R, commercially available from Cabot Corporation, US) is a synthetically prepared carbon black having a large surface area (20-300 m²/g) and electrical conductivity.

A solution of the above preformed and non-isolated cobalt-ligand complex is preferably absorbed onto the support material and the solvent removed. The pyrolysis step follows on from this, preferably under inert gas conditions. The thermal treatment may be carried out at 600-1100° C., wherein pyrolyses between 750 and 850° C. generate catalysts with particularly good catalytic activity and stability. Higher temperatures lead to a loss of catalytic activity, while lower temperatures mean lower stabilities. Depending on the nitrogen ligand selected, cobalt-containing particles are formed on the carbon-nitrogen surface.

The optimization of the catalyst treatment by pyrolysis in accordance with the invention has a significant influence on the activity and selectivity of the catalyst such that the catalytic material according to the invention may be used, surprisingly, for the hydrogenation of numerous aromatic nitro compounds. The hydrogenation of aromatic nitro compounds is preferably carried out at temperatures of 60 to 200° C., preferably at around 90 to 120° C.

Particular preference is given to using the catalysts supported on carbon with the formula Co-L/C in accordance with the invention for the hydrogenation of aromatic nitro compounds, where L has the abovementioned definition for L1 to L5. The use of 1,10-phenanthroline (L1) in particular led to a particularly selective and active system (Co-L1/C). However, 2,2′:6′,2″-terpyridine (L2) and 2,6-bis(benzimidazolyl)pyridine (L3) have also proven to be exceptionally suitable ligands. Whereas a ligand-free supported Co/C catalyst, and also materials in which only the organic nitrogen ligands were immobilized on the carbon material, did not lead to any desired product, the supported thermally-treated cobalt catalysts according to the invention show good to very good hydrogenation activity.

The substantial proportion of the aromatic amines obtained by the hydrogenation still comprise all multiple bonds on substituents of aromatic nuclei which were already present prior to the hydrogenation. Hence, the catalyst systems according to the invention are tolerant to all functional groups. Furthermore, they are cost-effective and environmentally friendly. They are therefore exceptionally suitable for industry for the selective hydrogenation of aromatic nitro compounds and lead either to corresponding aniline derivatives or, using heteroaromatic nitro compounds, to corresponding heteroaromatic amines, which are likewise valuable building blocks for the preparation of numerous agrochemicals and pharmaceuticals.

Besides tolerance to numerous substituents, the catalysts may be reused several times without loss of activity. They may easily be washed after each reaction and be dried overnight. For instance, the catalyst system Co-L1/C, for example, in the reaction of nitrobenzene to aniline, even in the 11th cycle, after 8 hours still showed a virtually complete conversion >99% and a yield of 98%.

A further advantage lies in that no protective gas techniques or drying agents are necessary and the reactions may be carried out with increasing water content. Interestingly, the reaction rate of the hydrogenation is dependent on the amount of water. For instance, the catalytic activity is significantly higher in pure water compared to THF. Surprisingly, the reaction time to reach complete conversion is prolonged when using dry organic solvents such as THF.

The invention also relates to novel supported cobalt-nitrogen ligand complexes with the formula Co-L/C, in which the support is a carbon support (C), which has been thermally treated at 750-850° C. The ligand L is selected from the group comprising:

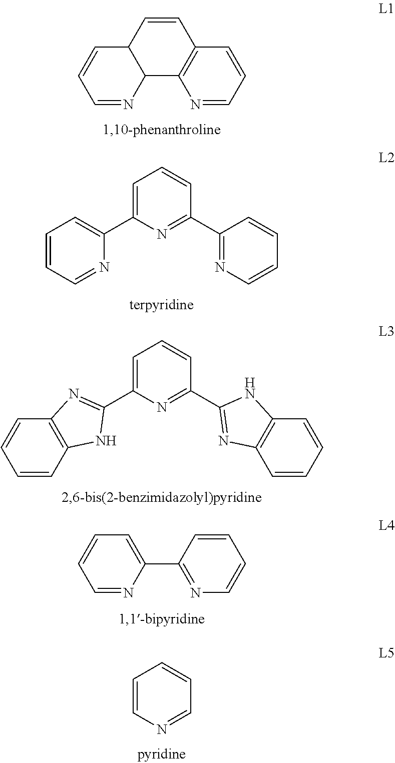 [SMILES: C1=CC2C=Cc3cccnc3C2N=C1 ; c1ccc(-c2cccc(-c3ccccn3)n2)nc1 ; c1cc(-c2nc3ccccc3[nH]2)nc(-c2nc3ccccc3[nH]2)c1 ; c1ccc(-c2ccccn2)nc1 ; c1ccncc1]

and also the 1,10-phenanthroline derivatives L1b: 2,9-dimethyl-4,7-diphenyl-1,10-phenanthroline, L1c: 4,7-dimethoxy-1,10-phenanthroline and L1d: 2,9-dimethyl-1,10-phenanthroline.

The invention is explained in more detail in the following working examples.

### EXAMPLES

### 1. Catalyst Preparation

Cobalt(II) acetate tetrahydrate (125 mg, 0.5 mmol) and 1,10-phenanthroline (180 mg, 1.0 mmol) (Co:phenanthroline=1:2 molar ratio) are stirred in ethanol (50 ml) for ca. 30 minutes at room temperature. After addition of carbon black (700 mg) (VULCAN® XC72R, Cabot Corporation Prod. Code XVC72R; CAS No. 1333-86-4), the reaction mixture is refluxed at 60° C. for 4 h. The reaction mixture is then cooled to room temperature and ethanol is removed under reduced pressure. The resulting solid is dried at 60° C. for 12 h and subsequently crushed to a fine powder. The thermal treatment (pyrolysis) of the powder is then carried out at 800° C. for 2 h under argon. Elemental analysis of Co-phenanthroline/C (wt %): C=92.28, H=0.20, N=2.70, Co=3.50, O=1.32

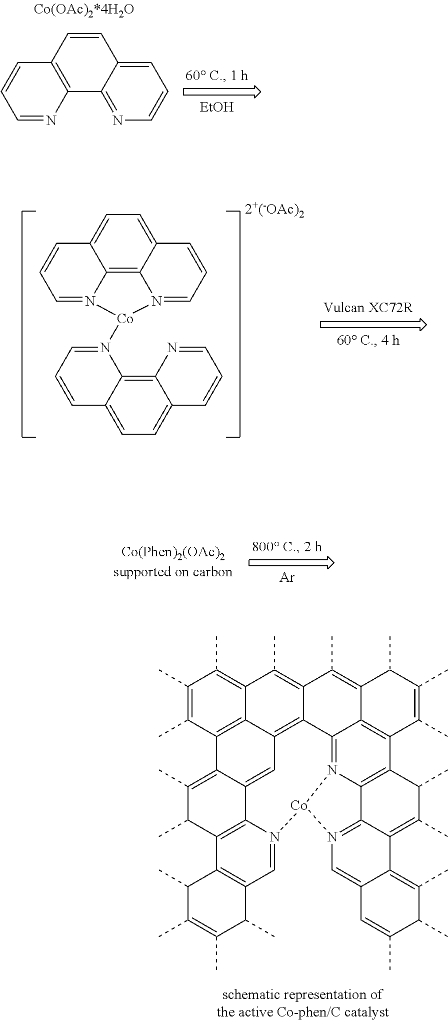

### 2. General Method for the Hydrogenation of Aromatic Nitro Compounds to the Corresponding Anilines

1 mol %, 3% by weight of Co-phenanthroline on carbon, 10 mg (Co-L1/C) prepared according to example 1, is placed in a glass reaction vessel equipped with a magnetic stirrer bar and a septum, likewise the respective aromatic nitro compound (0.5 mmol), the internal standard (hexadecane, 100 μl) and the solvent (THF, 2 ml) and H₂O (100 μl). The reaction vessel is placed in a 300 ml autoclave. Hydrogen is twice introduced into the autoclave and the reaction mixture is hydrogenated at 50 bar, the autoclave being placed in an aluminum block pre-heated to 110° C. During the reaction, the internal temperature measured in the autoclave is 104-106° C. After the reaction is completed, the autoclave is placed in a water bath and cooled to room temperature. Finally, the remaining hydrogen gas is discharged and samples are removed from the autoclave, washed with methylene chloride and analyzed by GC and GC-MS.

### 3. Catalyst Recovery

The reactions are carried out on a larger scale (5 mmol of aromatic nitro compound). An autoclave (100 ml) is filled with the cobalt catalyst (100 mg), THF (20 mL), hexadecane as internal standard (1 mL) and nitrobenzene (630 μL). Hydrogen is twice introduced into the autoclave and the mixture is hydrogenated at 60 bar. After each reaction, the catalyst is thoroughly washed with ethyl acetate and dried overnight under mildly reduced pressure.

### 4. Hydrogenation of Nitrobenzene Using Selected Supported Catalysts According to the Invention (Co-L1/C, Co-L2/C and Co-L3/C, Prepared According to Example 1) in Comparison to Co/C, L1/C, C, Fe-L1/C and Homogeneous Co-L1^{[a]}

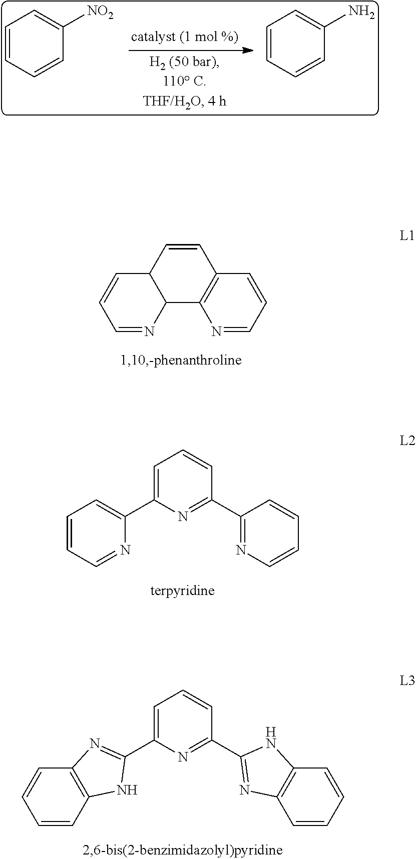 [SMILES: O=[N+]([O-])c1ccccc1 ; Nc1ccccc1 ; C1=CC2C=Cc3cccnc3C2N=C1 ; c1ccc(-c2cccc(-c3ccccn3)n2)nc1 ; c1cc(-c2nc3ccccc3[nH]2)nc(-c2nc3ccccc3[nH]2)c1]

TABLE 1

| | | Conversion | |
|---|---|---|---|
| No. | Catalyst | [%]^{[b]} | Yield [%]^{[b]} |
| 1 | Co/C | 5 | 0^{[e]} |
| 2 | L1/C | 1 | 0^{[e]} |
| 3 | C^{[f]} | 6 | 0^{[e]} |
| 4 | Co-L1/C^{[c]} | 100 | 95/99^{[a,d]} |
| 5 | Co-L1b/C^{[c]} | 100 | 95^{[a,d]} |
| 6 | Co-L1c/C^{[c]} | 100 | 92^{[a,d]} |
| 7 | Co-L1d/C^{[c]} | 68 | 56^{[a,d]} |
| 8 | Co-L2/C^{[c]} | 8 | 5^{[a]} |
| 9 | Co-L3/C^{[c]} | 26 | 19^{[a]} |
| 10 | Fe-L1/C | 15 | 1^{[e]} |
| 11 | Co-L1^{[g]} | 4 | 0^{[e]} |

^{[a]}Reaction conditions: 110° C., 4 h, 0.5 mmol of nitrobenzene, 1 mol % catalyst (3% by weight Co-L/C), 50 bar hydrogen, 2 ml of THF.
^{[b]}Determined by GC using n-hexadecane as internal standard.
^{[c]}M/L ratio 1.2.
^{[d]}Carried out in H₂O (3 ml).
^{[e]}Reaction time 16 h.
^{[f]}Pyrolized carbon.
^{[g]}Homogeneous catalyst.

The inventive catalyst system Co-L1/C has proven to be particularly reactive and selective. As is clear from Table 1 above, no aniline is formed in the presence of a homogeneous catalyst complex consisting of cobalt and phenanthroline (Table 1, No. 11), even if a 10-fold amount is used. Likewise, an iron catalyst analogous to the system according to the invention did not lead to any aniline formation (Table 1, No. 10).

### 5. Hydrogenation of Various Aromatic Nitro Compounds Using the Catalyst Co-L1/C (Prepared According to Example 1)

Table 2 shows the hydrogenation of substituted aromatic nitro compounds to industrially relevant anilines.

[a] Reaction conditions: 110° C., 0.5 mmol of aromatic nitro compound, 1 mol % catalyst (3% by weight of Co-phenanthroline on carbon), 50 bar hydrogen, 2 ml of THF, 100 μl of H₂O. [b] Determined by GC using n-hexadecane as internal standard. [c] Carried out in H₂O (3 ml). [d] Reduction selectively yields the diamine.

TABLE 2
Hydrogenation of substituted aromatic nitro compounds^{[a]}

| | Aromatic nitro | | Conversion | Yield |
|---|---|---|---|---|
| No. | compound | Time [h] | [%]^{[a,b]} | [%]^{[a,b]} |
| 1 | 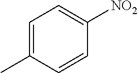 | 4 | >99 | 91/93^{[c]} |
| 2 | 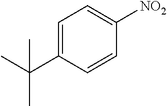 | 4 | >99 | 99/99^{[c]} |
| 3 | 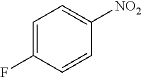 | 6 | >99 | 92 |
| 4 | 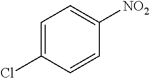 | 6 | >99 | 95 |
| 5 | 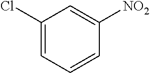 | 6 | >99 | 95/93^{[c]} |
| 6 | 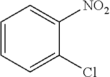 | 4 | >99 | 99 |
| 7 | 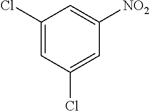 [SMILES: O=[N+]([O-])c1cc(Cl)cc(Cl)c1] | 6 | >99 | 97 |
| 8 | 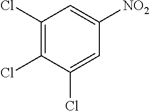 [SMILES: O=[N+]([O-])c1cc(Cl)c(Cl)c(Cl)c1] | 6 | >99 | 93/96^{[c]} |
| 9 | 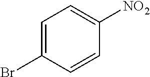 | 6 | >99 | 94 |
| 10 | 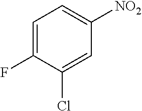 [SMILES: O=[N+]([O-])c1ccc(F)c(Cl)c1] | 6 | >99 | 83 |
| 11 | 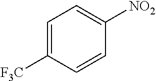 [SMILES: O=[N+]([O-])c1ccc(C(F)(F)F)cc1] | 6 | >99 | 93 |
| 12 | 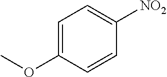 | 6 | >99 | 78 |
| 13 | 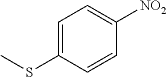 | 6 | >99 | 85/83^{[c]} |
| 14 | 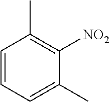 | 12 | >99 | 96 |
| 15 | 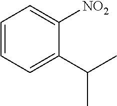 | 12 | >99 | 97/99^{[c]} |
| 16 | 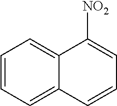 [SMILES: O=[N+]([O-])c1cccc2ccccc12] | 4 | >99 | 99 |
| 17 | 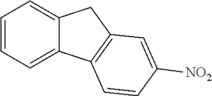 [SMILES: O=[N+]([O-])c1ccc2c(c1)Cc1ccccc1-2] | 12 | >99 | 91 |
| 18 | 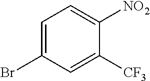 [SMILES: O=[N+]([O-])c1ccc(Br)cc1C(F)(F)F] | 4/12 | >99 | 78/88^{[c]} |
| 19 | 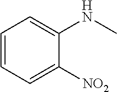 | 4 | >99 | 78 |
| 20 | 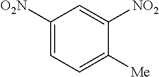 [SMILES: Cc1ccc([N+](=O)[O-])cc1[N+](=O)[O-]] | 12 | >99 | 83^{[c,d]} |

As is clear from Table 2 above, chloro- and fluoroanilines were obtained in good to excellent yields (83-99%; Table 2, No. 3-10). Aromatic nitro compounds with sterically demanding substituents (Table 2, No. 13-14), and also substrates bearing labile bromide substituents or sulfur, are also readily hydrogenated (Table 2, No. 9+13). Moreover, both electron-deficient substituents such as trifluoromethyl and electron-rich groups, e.g. methoxy and amino, are well tolerated.

### 6. Hydrogenation of Five Heteroaromatic Nitro Compounds Using Catalyst Co-L1/C

Reaction conditions: 110° C., 0.5 mmol of heterocyclic aromatic nitro compound, 1 mol % catalyst (3% by weight of Co-L1/C), 50 bar hydrogen, 3 ml of H₂O. The conversion and yield were determined by GC using n-hexadecane as internal standard. [c] Carried out in THF (2 ml) with 100 μl of H₂O.

Without further optimization, all substrates are converted in good yields (53-85%).

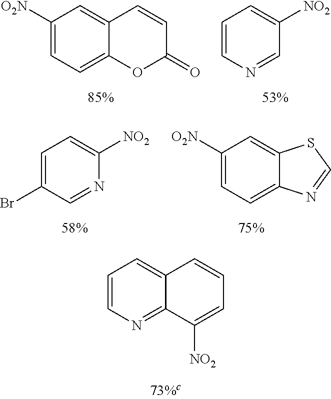

1. A process for hydrogenating aromatic nitro compounds which comprises using a thermally-treated supported cobalt as a catalyst for the hydrogenating, wherein the cobalt catalyst has been prepared by in situ immobilization of a cobalt-amine complex on an inorganic porous support and subsequent pyrolysis, and, in the cobalt-amine complex used, cobalt being present bonded to an aromatic or heterocyclic nitrogen ligand L, the nitrogen ligand being selected so as to form a polyaromatic structure with the cobalt atom.

2. The process as claimed in claim 1, wherein the nitrogen ligands of the cobalt-amine complex are selected from the group comprising phenanthrolines and derivatives thereof and pyridines, wherein the phenanthrolines have the following general formula 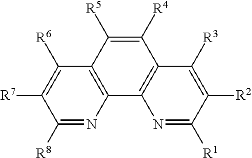
in which R¹ to R⁸ are identically or differently selected from the group comprising hydrogen, C1-C6-alkyl, C1-C6-Oalkyl, amino, carboxy, halogen, substituted and unsubstituted aryl, substituted and unsubstituted hetereoaryl.

3. The process as claimed in claim 1, wherein the nitrogen ligand L is selected from the group comprising the phenanthrolines L1: 1,10-phenanthroline and derivatives thereof, L1b: 2,9-dimethyl-4,7-diphenyl-1,10-phenanthroline, L1c: 4,7-dimethoxy-1,10-phenanthroline, L1d: 2,9-dimethyl-1,10-phenanthroline, L2: terpyridine, L3: 2,6-bis(benzimidazolyl)pyridine, L4: 1,1′-bipyridine and L5: pyridine.

4. The process as claimed in claim 1, wherein the inorganic support is composed of carbon (C), Al₂O₃ or TiO₂.

5. The process as claimed in claim 1, wherein the catalyst is a cobalt-amine complex supported on carbon with the formula Co-L/C, wherein L is selected from L1 to L5 as claimed in claim 3, the nitrogen ligand preferably being 1,10-phenanthroline (L1) and its derivatives 2,9-dimethyl-4,7-diphenyl-1,10-phenanthroline (L1b), 4,7-dimethoxy-1,10-phenanthroline, 2,9-dimethyl-1,10-phenanthroline (L1c).

6. The process as claimed in claim 1, wherein the catalyst used has been thermally treated at temperatures of 600 to 1100° C.

7. The process as claimed in claim 1, wherein the aromatic amine resulting from the hydrogenation still comprises all multiple bonds on substituents of aromatic nuclei which were already present prior to the hydrogenation.

8. The use as claimed in claim 1, wherein the hydrogenation of aromatic nitro compounds is carried out at temperatures of 60 to 200° C.

9. A supported cobalt-nitrogen ligand complex on a carbon support (C) with the formula Co-L/C, which has been thermally treated at 750 to 850° C., wherein the ligand L is selected from the group comprising L1: 1,10-phenanthroline and derivatives thereof, L1b: 2,9-dimethyl-4,7-diphenyl-1,10-phenanthroline, L1c: 4,7-dimethoxy-1,10-phenanthroline, L1d: 2,9-dimethyl-1,10-phenanthroline, L2: terpyridine, L3: 2,6-bis(benzimidazolyl)pyridine, L4: 1,1′-bipyridine and L5: pyridine.

10. A method for preparing a supported cobalt-nitrogen ligand complex on a carbon support with the formula Co-L/C as claimed in claim 9, wherein the solution of a cobalt salt is mixed with the nitrogen ligand L and the carbon support is then added and that, after optional removal of the solvent and drying, the pyrolysis is carried out.

11. The process as claimed in claim 2, in which R¹ to R⁸ are identically or differently selected from the group comprising H, Me, Et, OMe, NH₂, COOH, phenyl, F, CI and Br.

12. The process as claimed in claim 1, wherein the nitrogen ligand L is L1: 1,10-phenanthroline

13. The process as claimed claim 1, wherein the inorganic support is composed of carbon.

14. The process as claimed claim 2, wherein the inorganic support is composed of carbon (C), Al₂O₃ or TiO₂.

15. The process as claimed in claim 1, wherein the inorganic support is composed of carbon (C), Al₂O₃ or TiO₂.

16. The process as claimed in claim 2, wherein the catalyst is a cobalt-amine complex supported on carbon with the formula Co-L/C, wherein L is selected from L1 to L5 as claimed in claim 3, the nitrogen ligand preferably being 1,10-phenanthroline (L1) and its derivatives 2,9-dimethyl-4,7-diphenyl-1,10-phenanthroline (L1b), 4,7-dimethoxy-1,10-phenanthroline, 2,9-dimethyl-1,10-phenanthroline (L1c).

17. The process as claimed in claim 3, wherein the catalyst is a cobalt-amine complex supported on carbon with the formula Co-L/C, wherein L is selected from L1 to L5 as claimed in claim 3, the nitrogen ligand preferably being 1,10-phenanthroline (L1) and its derivatives 2,9-dimethyl-4,7-diphenyl-1,10-phenanthroline (L1b), 4,7-dimethoxy-1,10-phenanthroline, 2,9-dimethyl-1,10-phenanthroline (L1c).

18. The process as claimed in claim 4, wherein the catalyst is a cobalt-amine complex supported on carbon with the formula Co-L/C, wherein L is selected from L1 to L5 as claimed in claim 3, the nitrogen ligand preferably being 1,10-phenanthroline (L1) and its derivatives 2,9-dimethyl-4,7-diphenyl-1,10-phenanthroline (L1b), 4,7-dimethoxy-1,10-phenanthroline, 2,9-dimethyl-1,10-phenanthroline (L1c).

19. The process as claimed in claim 2, wherein the catalyst used has been thermally treated at temperatures of 600 to 1100° C.

20. The process as claimed in claim 3, wherein the catalyst used has been thermally treated at temperatures of 600 to 1100° C.
